(19) United States
(12) Patent Application Publication
(10) Pub. No.: US 2007/0105233 A1
(43) Pub. Date: May 10, 2007

(54) Canister piercer

(76) Inventors: Frank Chambers, Leicestershire (GB); Philip Wikeley, Worcestershire (GB)

Correspondence Address: WHITE & CASE LLP;PATENT DEPARTMENT, 1155 AVENUE OF THE AMERICAS, NEW YORK, NY, 10036, US

(21) Appl. No.: 10/579,161

(22) PCT Filed: Nov. 11, 2004
(86) PCT No.: PCT/SE04/01642

§ 371 Date: Aug. 14, 2006

(30) Foreign Application Priority Data
Nov. 14, 2003 (SE) 0303030-1

(51) Int. Cl. G01N 33/00

(52) U.S. Cl. 436/106

(57) ABSTRACT

This invention provides a method of analysing the contents of a pressurised container, such as a pressurised metered dose inhaler canister. This method comprises the steps of: enclosing said container in a pressure vessel and then pressurising the pressure vessel with a non-reactive fluid. The pressurised container within the pressure vessel is then pierced and the contents removed and analysed. The invention also consists of a device to carry out such a method.

### SUMMARY OF INVENTION

The invention relates to the removal of the contents of an MDI (metered dose inhaler) canister without causing the turbulence associated with this activity when conducted by the normal means. In doing so, the device allows the accurate determination of the amount of drug adhering to the inner side of the canister and/or the valve parts separately and in addition to the drug content of the expelled portion of the canister.

### BACKGROUND OF THE INVENTION

It is well known in the field of metered dose inhalers that some drugs adhere to the sides of the canister and onto valve parts in contact with the formulation. The formulation is usually in the form of a suspension of drug and/or excipients in a volatile propellant. Information on the amount of drug adhering to internal surfaces of the manufacturing equipment and in the pMDI is essential in the development of the product.

The most favoured way of alleviating this problem is to coat the inside of the can with a low energy coating such as those described in WO01/51222 and EP642992.

Drug adhesion is a significant problem in that it reduces the availability of the drug to the patient. Measurement of the parameter of potential drug adhesion allows different coatings to be explored and makes calculations of the drug overage to be accurately ascertained. The manufacturing overage is calculated using the concentration of the drug in the manufacturing vessel. An overage is the extra drug which has to be added to the formulation during manufacture to account for that which may adhere to the manufacturing vessel and the inside of the canister over the time it is stored prior to reaching the patient. Regulatory bodies generally require an overage of less than 10%. Accurate determination of loss of drug during manufacture and on the wall of the canister are therefore essential.

Also, the tendency for the pMDI to be stored inverted, i.e. valve down, inside the actuator exacerbates the problem as the formulation is in contact with the valve for a significant time giving the possibility of adhesion of formulation to the valve components.

The potential risks of a high can overage comes from the possibility of reversibility, where the excess drug falls back into suspension and results in a higher than required dose of drug delivered to the patient. The dose uniformity testing carried out during batch testing may determine this but would result in batch failure.

It is imperative, therefore, that the amount of drug adhesion (if any) is determined at an early stage of the MDI development. The normal procedure of cooling the can, piercing it and pouring out the contents is not accurate as the cooling itself can cause deposition (as a result of propellant evaporation) or cracking of the deposit, (in the latter, causing it to drop into the main body of the formulation), each in turn giving a falsely high or low result.

Piercing the can and allowing the contents to escape under pressure, is not a viable alternative either as the drug may be deposited as the propellant evaporates or is disturbed and removed during the uncontrolled exit of the propellant. Therefore, it can be seen that current methods for determining the amount of can deposition are not reproducible and are not suitable for low dose products as small losses in the method become unacceptable when small amounts are to be recovered i.e. micrograms. There are several can piercing applications in the prior art, for example, U.S. Pat. No. 3,828,976. However the majority are for emptying of the can per se, either for capture of the propellant or recycling of the can material and are not intended to be a vehicle by which the can and contents are analysed.

U.S. Pat. No. 6,393,900 details an apparatus for emptying the contents of an MDI and capturing the contents for analysis. This is essentially an automated can contents analysis and gives total can concentrations. The invention does not use a pressurised system and is not designed to measure drug adhesion inside the canister as it only takes into account the total of the can contents. The contents are forced out of the canister under its own pressure; there is no effort to control it only to contain it. The invention detailed in this application allows a differential analysis of the can contents, including the proportion of drug left inside the canister and, (if the valve is detached before the empty canister is washed), a measurement of drug on can and valve separately.

Another patent, DE20203999, details an apparatus which pierces the canister and confines the contents for analysis but the canister is not pre-pressurised and the primary objective is to empty the can into a receptacle without having to cool it first and to use an ion-selective electrode to determine a required parameter under pressure while the contents are held within the receiving vessel. There is no attempt to measure contents left inside the canister.

Therefore, it is the purpose of this invention to control the exit of the contents of the canister from said canister in a controlled manner such that the layer of drug adhering to the inside of the can and on the valve mechanism may be left behind unperturbed and therefore accurately measured.

### DESCRIPTION OF THE INVENTION

One form of the present invention may broadly be said to consist in a method of analysing the contents of a pressurised container comprising the steps of: enclosing said container in a pressure vessel; pressurising said pressure vessel with a non-reactive fluid; piercing said pressurised container within said pressure vessel; and analysing the content of said pressurised container when drawn off through said pierce ring.

The term fluid is used in accordance with the definition in the Oxford English dictionary where by a fluid is “a substance, as a gas or liquid, that is capable of flowing freely”.

Preferably the pressurised container is a canister container medicament.

Preferably said canister is a metered dose inhaler canister.

Preferably said non-reactive fluid is nitrogen. The nitrogen is preferably gaseous nitrogen.

Propellant, for example as a pressurised gas or vapour, can be used instead of nitrogen. The propellant can, for example, be a HFA propellant. One advantage of using propellant is that a more efficient mixing is achieved.

The apparatus is a rig for piercing an MDI canister in order to empty it of its contents without disturbing any material adhered to the surface of the can.

If rapid emptying took place, i.e. as pressure is suddenly released, the can deposition would be disturbed. In this instance the can is pre-pressurised by being pierced in the side, initially by a cannula under pressure by nitrogen.

As the base is then pierced, the pressure inside the can is kept constant allowing the can contents to flow out of the base of the canister with control, thereby not disturbing any material deposited on the sides or on the valve.

Obstacles to be overcome in the design of the invention were:

Designing a vessel which could hold a pressurised canister securely enough to pierce it using external controls without a leak or loss of can contents into the holding vessel.

Minimising losses in the transfer line linking the piercer with the collection vessel.

To validate the wash efficiency to ensure full recovery of deposited and evacuated drug.

Devise a means of evacuating the collection vessel of propellant without losing drug content.

To pierce the canister and provide a pressurised supply to the inside of the can, prior to piercing at the base

To be able to determine accurately the drug left on the can wall separately from the rest of the can contents.

The following apparatus was devised to overcome these obstacles:

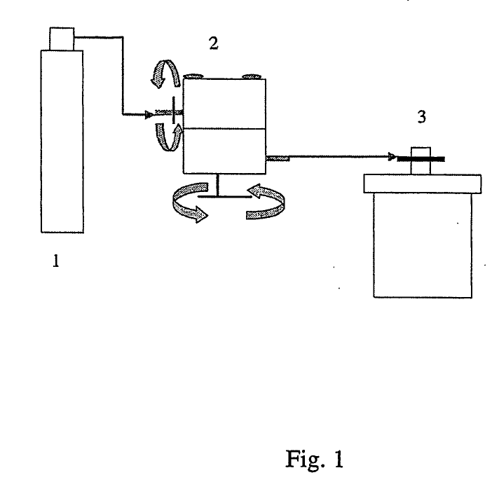

As shown in FIG. 1, the device comprises several components:
a) a pressuriser comprising a nitrogen supply 1, control gauge and connecting tube
b) a collection vessel comprising three sections; (i) the main body and lid (ii) a three-way valve on the top (iii) a nitrile seal between the base and lid to contain and maintain pressure until venting is needed.
c) A two compartment device which constitutes the main part of the invention comprising two halves joined together by three screws, with an internal void of suitable size to accommodate the pMDI canister. The pMDI fits in tightly and is sealed in place by three rubber O-rings. The 3 screws seal the apparatus when tightened.

The top section has a rotating bar attached at the side. When rotated the piercer enters the space occupied by the pMDI canister. The piercing action is similar to a needle. Once the can is pierced the nitrogen pressure is applied via the connecting tube. The bottom section has a piercer at the base and with the aid of a second rotating bar; this pierces the base of the canister and is then retracted.

The base has a hole at the bottom corner, which is so designed to channel the product via the transfer line under pressure from the nitrogen, into the collection vessel.

Method of Sample Collection to determine Drug adhesion.

To summarise, the method of collection is as follows:
Weigh the can and vessel
Seal can into piercer
Pierce side of can allowing nitrogen to enter at required pressure
Pierce base of can and retract
Open 3 way valves allowing product to flow into collection vessel
Leave nitrogen flowing for several minutes
Seal vessel and can piercer using 3 way valves
Reweigh can and vessel
Vent into dose unit (only on validation)
Wash vessel with solvent to dissolve drug and excipients
Collect and analyse drug remaining in the can and on valve.

The wash method will be determined by the product and would be determined as a normal part of validation of the recovery of the drug from the system and is well within the knowledge of a competent person skilled in analytical chemistry. The drug content assay would also constitute normal practice as part of the usual product development process and within the scope of a skilled person. Therefore it is perfectly conceivable for this method to be adapted for other formulations beyond the examples given and is within the scope of a skilled person to do that using this method, as the method is not dependent on the type of materials used in the formulation, the canister or the coating.

### EXPERIMENTAL DETAILS

The formulation under test was a low concentration suspension formulation of formoterol fumarate dihydrate (FED). The drug was suspended in a blend of propellants HFA 134A and HFA 227. The description of the formulation is contained in patent application WO03/63843.

The main concerns of the experimental validation were a) leakage b) recovery efficiency. Parameters affecting recovery were: efficiency of transfer along the transfer line linking the can piercing unit with the recovery vessel, the vessel washing procedure, potential losses on venting the collection chamber when venting into a dose collection vessel and subsequent washing of that vessel, and recovery of the deposits of drug inside the vented canister.

Placebo cans were used to check for carryover of FFD from one can to the next. These were analysed between cans containing active formulation to check for carry over between cans. Negligible amounts of FFD were found using the placebo cans showing that carryover of drug was not a concern and that the wash method was efficient.

Early trials resulted in low recovery due to leakage of the O-rings, which sealed the vessels with subsequent loss of drug. Care must be taken to align the O-rings correctly and seal the vessel. Any damaged O-rings must be replaced immediately.

Also early trials detected drug in the can piercer as a result of leakage around the cannula. Due to the presence of drug in the piercer it was decided to lower the nitrogen pressure prior to piercing, thus reducing or eliminating the tendency for FFD to be forced into the piercer. Additionally, the objective was to create a pressurised equilibrium between the can and device. Also, the lower pressure would reduce the chance of the O-rings leaking.

To this end the pressure was lowered from 7 bar to 4 bar and the nitrogen left flowing for 1 minute into the piercing device prior to piercing the can. The effect was to reduce the FFD deposited in the piercer.

### Summary of the Experimental Validation of the Device

1. Placebo cans, used to check for carry over of drug from one can to another, showed that there was negligible drug deposited in the transfer line.

2. When the pressurised vessel was vented into a dose delivery unit on venting to test for loss of drug during the venting process, the resulting wash solution showed no traces of drug.

3. The wash procedure for the collection vessel used three washes. Negligible amounts of drug were found in the second and third washes, meaning a single wash could be used. (This, of course, may vary from drug to drug)

4. The can piercer was analysed for FF) deposits. Initially it was found to contain a significant amount of drug. However allowing the piercer device to pressurise for 1 minute prior to piercing and lowering the pressure (thereby reducing the pressure differential between can and device) eliminated this problem. To be certain of full recovery of the FFD, this was washed as well.

The validation and optimisation of the method resulted in an acceptable recovery of FFD, such that quantisation of can deposition could be determined reliably. Results of cans analysed by the resulting method can be seen in Table 1 and the method is outlined below:
Weigh the can and vessel
Seal can into piercer as described previously
Leave nitrogen flowing at required pressure for 1 minute
Pierce side of can allowing nitrogen to enter at required pressure
Pierce base of can and retract
Open 3 way valves allowing product to flow into collection vessel
Leave nitrogen flowing for several minutes
Seal vessel and can piercer using 3 way valves
Reweigh can and vessel
Vent collection vessel
Wash vessel and can piercer with solvent to dissolve drug and excipients
Collect and analyse drug remaining in the can and on valve.
   Calculations used in Tables 1 and 2
A=Residual drug in Can (% w/w)
B=Vessel Washings (% w/w)
C=Can Piercer Washings (% w/w)
D=A+B+C=Total FFD Recovered from can and piercer components (% w/w)
E=Total recovery=D/F×100 (%)

Where F is the expected total can content (from QA batch testing data). For uncoated cans using 134A, expected total can content was 0.0216%w/w and for blend formulation in coated cans it was 0.0167% w/w.

TABLE 1
results of cans tested using the system described above. Drug in uncoated can in HFA134a
DRUG IN UNCOATED CAN IN HFA134A

| | Residual drug | Vessel | Can Piercer | Total FFD | | |
|---|---|---|---|---|---|---|
| Can | in Can % w/w | Washings % w/w | Washings % w/w | Recovered % w/w | Recovery % | Conditions |
| 1 | 0.0086 | 0.009 | 0.0018 | 0.0194 | 90% | 4 bar pressure |
| 2 | 0.0080 | 0.0101 | 0.0019 | 0.0200 | 93% | 4 bar pressure |
| 3 | 0.0117 | 0.0075 | 0.0019 | 0.0211 | 98% | 7 bar pressure |
| 4 | 0.0114 | 0.0086 | 0.0019 | 0.0219 | 101% | 7 bar pressure |
| 5 | 0.0096 | 0.0097 | 0.0019 | 0.0212 | 98% | 4 bar pressure |
| 6 | 0.0106 | 0.0039 | 0.004 | 0.0185 | 86% | 4 bar pressure |
| 7 | 0.0092 | 0.0102 | 0.0023 | 0.0217 | 100% | 4 bar pressure |
| 8 | 0.0098 | 0.0069 | 0.0027 | 0.0194 | 90% | 4 bar pressure |
| 9 | 0.0096 | 0.0097 | 0.0015 | 0.0208 | 96% | 4 bar pressure |

TABLE 2
Results
Drug in coated can in HFA 227/134A blend

| | Residual drug | Vessel | Can Piercer | Total FFD | | |
|---|---|---|---|---|---|---|
| Can | in Can % w/w | Washings % w/w | Washings % w/w | Recovered % w/w | Recovery % | Conditions |
| 1 | 0.0037 | 0.0095 | 0.0028 | 0.016 | 96% | 4 bar pressure |
| 2 | 0.0027 | 0.0094 | 0.0023 | 0.0144 | 86% | 4 bar pressure |
| 3 | 0.0028 | 0.0056 | 0.0044 | 0.0128 | 77% | 4 bar pressure |
| 4 | 0.0025 | 0.0128 | 0.0018 | 0.0171 | 102% | 4 bar pressure |
| 5 | 0.0024 | 0.0071 | 0.0042 | 0.0137 | 82% | 4 bar pressure |

The most noticeable difference in the two batches is between the uncoated and coated cans: the deposition in the uncoated can having on average 0.0100 w/w % deposition and the average on the coated can was 0.0028 w/w %, this represents a significant effect when using a coated can. I.e. the can deposition in the 134A/uncoated can is, on average, 45% compared to 17% in the blend/coated can, when compared with the total Formoterol content in the formulation.

It is entirely possible that the device and method would therefore distinguish between different formulation deposits using the same coating or the same formulation exposed to different coatings. The invention is not limited to the formulations or canisters described here and could easily be applied to other cans of differing size by altering the dimensions of the can holding vessel.

The conclusions which can be drawn from the results in Tables 1 and 2 is that:

The device is suitable for emptying can contents controllably such that any drug deposition on the canister wall is left undisturbed and can be analysed separately.

The method is accurate and reproducible and can be used to ascertain the differences between deposits of drug in different environments such as uncoated and coated surfaces or between formulations.

Separation of can and valve allows evaluation of deposition in each component.

1. A method of analysing contents of a pressurised container the method comprising the steps of:
enclosing said container in a pressure vessel;
pressurising said pressure vessel with a non-reactive fluid;
piercing said pressurised container within said pressure vessel; and
analysing the contents of said pressurised container when drawn off through said piercing.

2. The method according to claim 1, wherein said piercing is performed by the following steps:
forming a first piercing, by which said first piercing the pressurised container is pre-pressurised, and
forming a second piercing,
wherein the pressure inside the pressurised container is kept constant by said first piercing when the contents of the pressurised container are drawn off through said second piercing.

3. The method according to claim 1, wherein the pressurised container comprises a canister wall. and the method further comprises the step of analysing contents remaining on the canister wall after contents of the container are drawn off.

4. The method according to any one of claims 1, 2 or 3, wherein said container is a metered dose inhaler canister.

5. The method according to any one of claims 1, 2 or 3, wherein said non-reactive fluid is a propellant.

6. The method according to any one of claims 1, 2 or 3, wherein said non-reactive fluid is nitrogen.

7. The method claim 6, wherein said nitrogen is gaseous.
